(19) United States
(12) Patent Application Publication
(10) Pub. No.: US 2004/0059403 A1
(43) Pub. Date: Mar. 25, 2004

(54) Suture sleeve

(75) Inventor: Mary-Helene Massullo, Brookfield, OH (US)

Correspondence Address: HAHN LOESER & PARKS, LLP, TWIN OAKS ESTATE, 1225 W. MARKET STREET, AKRON, OH, 44313, US

(73) Assignee: Geriche, Inc.

(21) Appl. No.: 10/253,238

(22) Filed: Sep. 24, 2002

(51) Int. Cl. A61N 1/05

(52) U.S. Cl. 607/119

(57) ABSTRACT

An inflatable suture sleeve used to secure a pacer lead of a pacer system to adjacent tissue. The suture sleeve has an internal cavity, which, preferably, has an inflatable balloon therein. The suture sleeve is positioned as desired and is sutured or stapled to tissue within the patient's chest. An inflation syringe is attached to an inflation nipple on the suture sleeve. Then a predetermined quantity of saline solution is injected into the balloon expanding the balloon so that internal ridges, on a flexible bore through which the pacer lead extends, tighten against the pacer lead to securely hold the pacer lead to the suture sleeve. Inflatable suture sleeve secures the pacer lead as the balloon causes pressure and a snug fit against the pacer wire when inflated. When not inflated, suture sleeve can be re-positioned on pacer wire.

### BACKGROUND OF THE INVENTION

This invention relates generally to suture sleeves for anchoring the lead bodies of implantable medical devices such as cardiac pacemakers, and more particularly to a suture sleeve assembly for securely gripping and anchoring a lead body without damage thereto.

During the implantation of an endocardial lead body, the lead is introduced into the heart using a venous approach, usually from the subclavian or cephalic vein in the shoulder area under the pectoral muscle. To stabilize the lead body at the venous entry site, the lead body is secured to both the vein and to the surrounding fascia tissue. A suture placed around the vein near the lead entry point ties the lead body to the vein, and a suture sleeve around the lead body is used to anchor the lead body to adjacent tissue.

Suture sleeves in present use are generally tubular structures molded out of a soft, implantable elastomer, such as silicone. After the lead body is tied to the vein, the sleeve is slid along the lead body to the location at which the lead is to be anchored to the underlying tissue. One or more sutures are then tied around the sleeve to compress it and thereby secure it to the lead body. Circumferential grooves in the outer surface of the sleeve are typically provided for this purpose. The last step is to anchor the sleeve to adjacent body tissue; sutures passed through eyelets formed in a pair of tabs projecting from the sleeve provide the required anchoring.

These existing suture sleeves have several drawbacks. For example, it is difficult for the physician to control the degree to which these sleeves are compressed when they are secured to the lead body. The ligature around the sleeve must be tight enough to prevent the lead body from sliding in the suture sleeve but not so tight as to damage the insulation of the lead body. This is especially important with bipolar coaxial leads because an excessively tight ligature could rupture the lead insulation and cause the outer and inner electrical leads to come into contact with each other, resulting in a short circuit. Over tightened ligatures can also result in electrical lead fractures. These problems are common enough to warrant the inclusion of cautionary information in pacemaker product literature or in notices included in the product packaging regarding the use of anchoring sleeves. It would therefore be desirable to eliminate the need for sutures for compressing the sleeve.

The foregoing illustrates limitations known to exist in present suture sleeves. Thus, it is apparent that it would be advantageous to provide an alternative directed to overcoming one or more of the limitations set forth above. Accordingly, a suitable alternative is provided including features more fully disclosed hereinafter.

### SUMMARY OF THE INVENTION

In one aspect of the present invention, this is accomplished by providing a suture sleeve comprising: a tubular body having a flexible bore extending longitudinally therethrough and having an expandable closed annular space circumferentially extending at least partially around the bore.

In another aspect of the present invention, this is accomplished by providing a suture sleeve comprising: a tubular body having a bore extending therethrough and an internal cavity circumferentially extending partially around the bore; and an inflatable balloon within the tubular body internal cavity.

In another aspect of the present invention, this is accomplished by providing a suture sleeve comprising: a reinforced silicon tubular body having: a flexible bore extending therethrough; an internal cavity circumferentially extending partially around the bore; and a plurality of attachment wings extending outward from the tubular body, the bore having a plurality of inwardly extending ridges thereon, the plurality of inwardly extending ridges comprising pairs of opposed arcuate ridges, each arcuate ridge circumferentially extending less than 180°, a pair of arcuate ridges being longitudinally spaced from an adjacent pair of arcuate ridges, a central portion of an undersurface of the tubular body being substantially flat; and an inflatable balloon within the tubular body internal cavity, the balloon having a self-sealing inflation port, the inflation port extending through an external surface of the tubular body.

In another aspect of the present invention, this is accomplished by providing a method of anchoring a lead body of an implantable surgical device comprising the steps of: providing a suture sleeve having a through bore and having an inflatable lead body engaging portion, the lead body extending through the suture sleeve bore; securing the suture sleeve to the desired tissue; and inflating the inflatable lead body engaging portion of the suture sleeve to engage the suture sleeve with the lead body.

In another aspect of the present invention, this is accomplished by providing a surgical kit comprising: an inflatable suture sleeve having a bore extending therethrough and an inflatable chamber at least partially surrounding the bore; a syringe; and a predetermined amount of saline solution for filling the suture sleeve inflatable chamber.

The foregoing and other aspects will become apparent from the following detailed description of the invention when considered in conjunction with the accompanying drawing figures.

### BRIEF DESCRIPTION OF THE DRAWING FIGURES

FIG. 1 is a front elevational view of a human upper torso of a patient and shows a pacer lead extending between a pacer and a heart with the lead being fixed by a suture sleeve of the present invention to the patient's skin;

FIG. 2 is a top view of a suture sleeve according to the present invention;

FIG. 3 is a side view of the suture sleeve shown in FIG. 2;

FIG. 4 is a cross-sectional side view of the suture sleeve taken on line 4-4 of FIG. 2;

FIG. 5 is an end view of the suture sleeve shown in FIG. 2;

FIG. 6 is a cross-sectional view of the suture sleeve taken on line 6-6 of FIG. 2;

FIG. 7 is a schematic cross-sectional view of a portion of the suture sleeve shown in FIG. 2 illustrating the clamping of the pacer lead;

FIG. 8 is an exploded perspective of an inflation nipple on the suture sleeve shown in FIG. 2 and an inflation syringe;

FIG. 9 is side view of an inflation syringe for use with the suture sleeve shown in FIG. 2;

FIG. 10 is a side view of a staple for use with the suture sleeve shown in FIG. 2;

FIG. 11 is a schematic view of a portion of the suture sleeve shown in FIG. 2, showing the attachment of the suture sleeve with a staple to tissue; and

FIG. 12 is a perspective view of a surgical stapler for use with the suture sleeve shown in FIG. 2.

### DETAILED DESCRIPTION

FIG. 1 shows a pacer system 10 including a pacer 12 adapted to be inserted in a pocket on the chest 14 of a patient and a pacer lead 16 having a tip electrode assembly 18 implanted within the patient's heart 20. The pacer lead 16 extends from the pacer 12 to a position or area 22 where the pacer lead 16 extends into and through a vein to the heart 20 where the tip electrode assembly 18 is positioned in a chamber of the heart 20 for pacing of the heart 20.

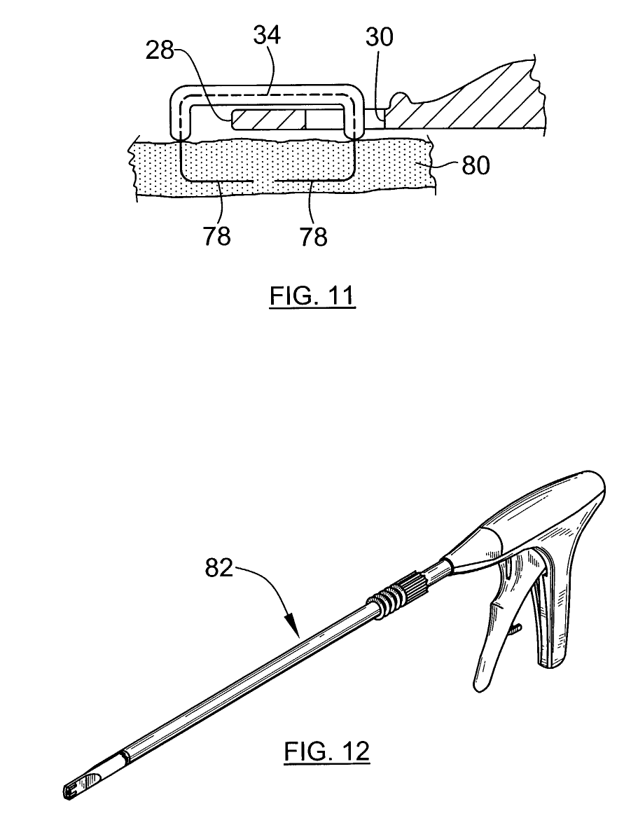

Once the tip electrode assembly 18 of the pacer lead 16 is appropriately positioned, an elastomeric suture sleeve 24, which has been positioned on the lead 16 and, which is movable thereon, is moved along the lead 16 to a location where it is desirable to fasten the lead 16 to underlying tissue 80 (See FIG. 11). In FIG. 1, the suture sleeve 24 is fastened to underlying tissue in the area 22 near where the pacer lead 16 exits the vein.

Suture sleeve 24 can be used with other types of implantable surgical devices in addition to pacer systems.

Briefly, the present invention consists of an inflatable suture sleeve 24. The suture sleeve 24 has an internal cavity 25, which, preferably, has an inflatable balloon 46 therein. The suture sleeve 24 is positioned as desired and is sutured or stapled to tissue 80 within the patient's chest 14. An inflation syringe 60 is attached to an inflation nipple 36 on suture sleeve 24. Then a predetermined quantity of saline solution 74 is injected into balloon 46 expanding the balloon so that internal ridges 40, on a flexible bore 38 through which pacer lead 16 extends, tighten against pacer lead 16 to securely hold the pacer lead 16 to the suture sleeve 24. Inflatable suture sleeve 24 secures pacer lead 16 as balloon 46 causes pressure and snug fit against pacer wire 16 when inflated. When not inflated, suture sleeve 24 can be re-positioned on pacer wire 16, as do prior art suture sleeves.

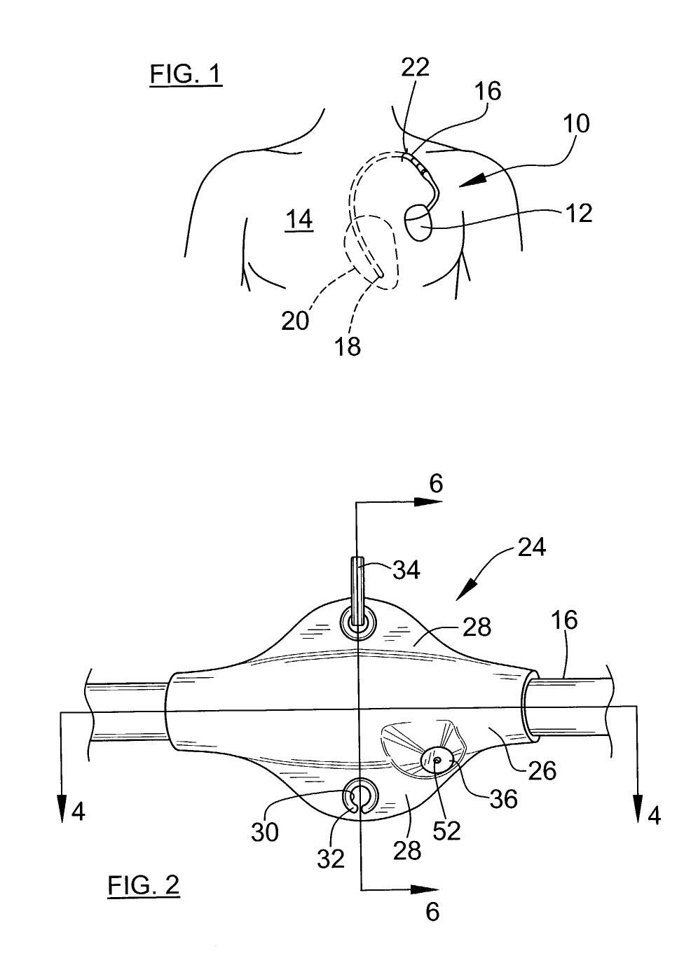

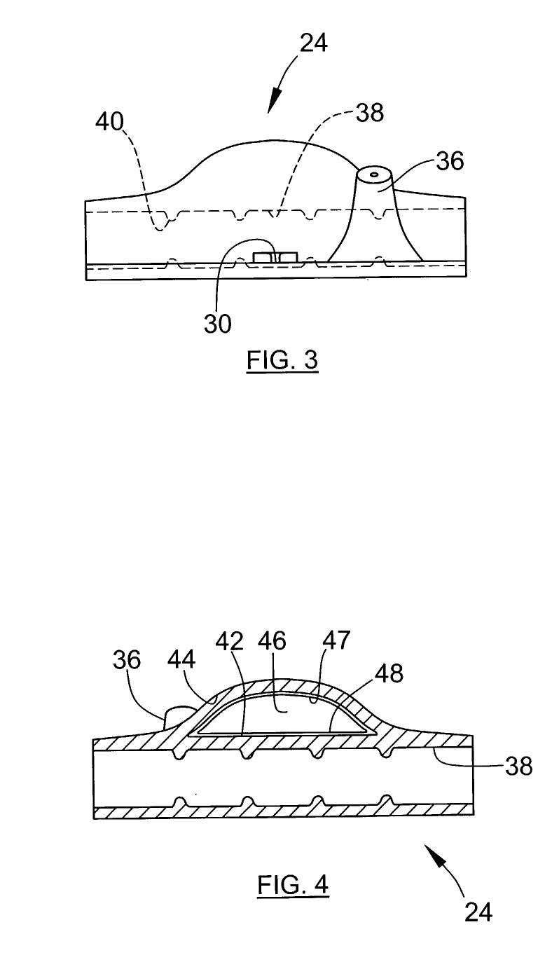

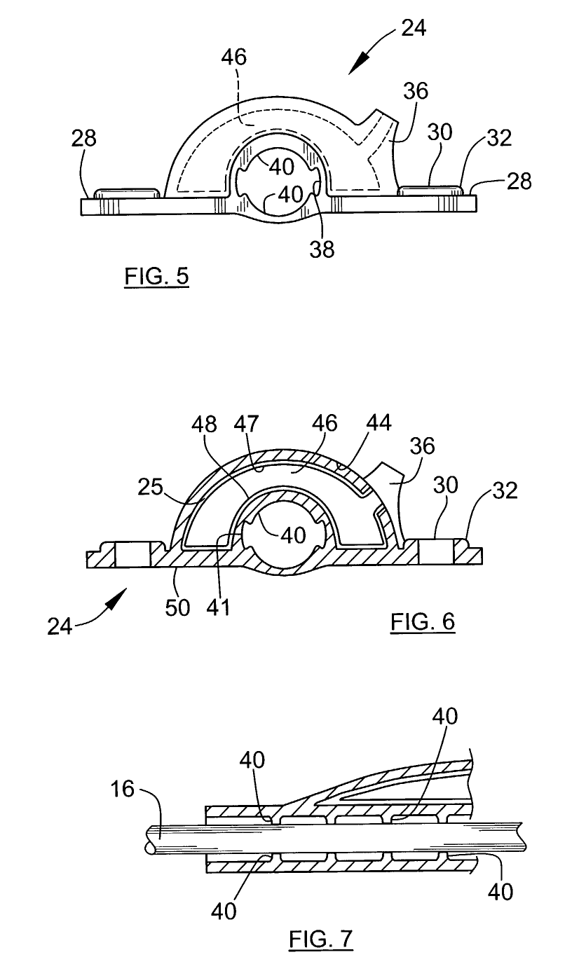

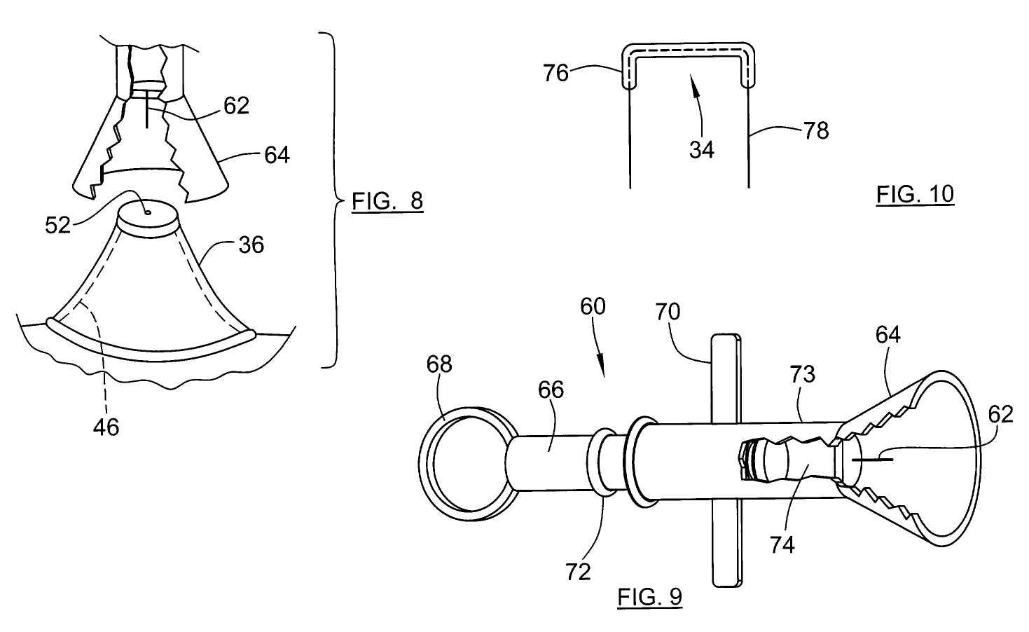

FIGS. 2 through 7 show the suture sleeve 24 of the present invention. Suture sleeve 24 has a flexible tubular central body 26 with a flexible bore 38 extending lengthwise therethrough. Pacer lead 16 fits through the flexible bore 38. Suture sleeve 24 has a pair of attachment wings 28 extending laterally from the tubular body 26. Each attachment wing 28 has at least one staple/suture hole 30 to allow the suture sleeve 24 to be sutured or stapled to the surrounding tissue 80. A raised guide ring 32 extends almost all the way around staple/suture hole 30 to guide the placement of a staple 34 as shown in FIG. 2.

The interior of suture sleeve 24 has an internal cavity or closed annular space 25 that extends circumferentially around flexible bore 38. Preferably, the internal cavity extends only partially around flexible bore 38 as shown in the FIGURES. FIG. 4 shows the internal cavity exterior wall 44 and the internal cavity interior wall 42. Preferably, an inflatable balloon 46 is positioned within this internal cavity. Balloon 46 also has an outer wall 47 proximate the internal cavity exterior wall 44 and an inner wall 48 proximate the internal cavity inner wall 42. This arrangement provides four layers, internal cavity exterior wall 44, internal cavity interior wall 42, balloon inner wall 48 and balloon outer wall 47, against leakage of the saline solution 74 used to inflate the balloon 46. Although it is preferred to use balloon 46, suture sleeve 24 could be used without the balloon 46. In this event, the saline solution 74 would be injected into the internal cavity 25 to inflate the suture sleeve 24.

Balloon 46 can be fixed to the interior surface of the suture sleeve internal cavity or it can be positioned within the cavity and be free to move relative to the internal cavity surfaces. Suture sleeve 24 has an inflation nipple 36 with a self sealing port 52 therein. Balloon 46 extends upward into inflation nipple 36 and is in fluid communication with self sealing port 52. Self sealing port 52 is similar to an IV port or lock. Preferably, inflation nipple 36 extends up and away from pacer bore 38 so that insertion of needle 62 does not inadvertently penetrate balloon 46 (other than through self sealing port 52) or pacer lead 16.

Preferably, the interior of flexible bore 38 has a plurality of inwardly projecting ridges 40 to more tightly engage the pacer lead 16. Ridges 40 are arranged as a plurality of longitudinally spaced pairs (See FIGS. 4 through 7). Each pair consists of opposed rings that extend partially around the inner circumference of flexible bore 38. Preferably, each ring extends less than 180° around the inner circumference so that flexible bore 38 can compress against pacer lead 16. The wall areas 41 of flexible bore 38 are thin, compared to the thickness of ridges 40, so that bore 38 is flexible and will compress against pacer lead 16 when balloon 46 is inflated.

Preferably, suture sleeve 24 is flexible so that the internal cavity can expand and contract with balloon 46. However, suture sleeve 24 can be less flexible or semi-rigid so that balloon 46 expands and contracts independently of the internal cavity.

Suture sleeve 24 is preferable formed from reinforced silicon. The preferred reinforcement is a nylon net or mesh to add strength to suture sleeve 24 and to prevent tears. Additional reinforcement can be added around the suture/staple holes 30. Suture sleeve 24 has a smooth contour to avoid adding more surface features conducive to infection. The bottom 50 (or at least a central portion of the bottom) of suture sleeve is preferably flat, to facilitate attachment to tissue 80.

Reinforcement or guide ring 32 along with inflation nipple 36 can be colored to help visualize the suture or staple placement or the inflation syringe needle insertion.

FIG. 9 shows an inflation syringe 60 for use with suture sleeve 24. Syringe 60 has a barrel 73 filled with a predetermined amount of saline solution 74. Alternatively, saline solution 74 could be provided separately and then drawn into barrel 73. A plunger 66 fits into barrel 73, which, when pressed, expels saline solution 74 out of syringe 60. A needle 62 is provided on the end of barrel 73. A guide cone 64 surrounds needle 62 to protect needle 62 and to guide needle 62 into self sealing port 52 in inflation nipple 36. Since inflation nipple 36 preferably extends upward and away from the outer surface of suture sleeve 24, this helps prevent inadvertent insertion of needle 62 into suture sleeve 24 at locations other than inflation nipple 36. Guide cone 64 also helps to prevent inadvertent injury from needle 62. To facilitate handling of syringe 60, a pair of finger supports 70 project from opposite side of barrel 66. These supports 70 are intended for middle and index fingers. A thumb ring 68 is formed at the external end of plunger 66. Plunger 66 includes a stop 72 projecting from plunger 66 to prevent the plunger from being pushed too far into barrel 73 and injecting more than the predetermined quantity of saline solution 74 into balloon 46.

Syringe 60 can used to inflate balloon 46 prior to insertion of pacer 12 into a patient to assure that suture sleeve 24 will properly inflate and tighten against pacer lead 16. After checking proper operation of suture sleeve 24, plunger 66 is retracted while needle 62 is still inserted into inflation nipple 36 to withdraw saline solution 74 from balloon 46 to deflate balloon 46.

FIGS. 10 and 11 show a surgical staple 34 for use with suture sleeve 24. The central portion of staple 34 and the upper portions of ends or legs 78 are coated with silicon 76 to protect suture sleeve 24 and to help secure staple 34 to suture sleeve 24. A typical single use surgical stapler 82 can be used to secure suture sleeve 24 to tissue 80.

Normally, suture sleeve 24 is pre-attached to pacer lead 16. Typically, pacer leads 16 have a variety of fittings and connections that would prevent suture sleeve 24 from being attached to pacer lead 16 after pacer lead 16 assembly. Preferably, suture sleeve and pacer lead is packaged as a kit containing a pacer system 10 with pacer lead 16 and attached suture sleeve 24, inflation syringe 60 with predetermined quantity of saline solution 74, surgical staples 34 and surgical stapler 82 along with other needed or preferred items.

When being installed, suture sleeve 24 can be attached to tissue 80 and then inflated or can be inflated prior to attachment to tissue 80.

Having described the invention, what is claimed is:

1. A suture sleeve comprising:
a tubular body having a flexible bore extending longitudinally therethrough and having an expandable closed annular space circumferentially extending at least partially around the bore.

2. The suture sleeve according to claim 1, wherein the closed annular space has an inflation port therein.

3. The suture sleeve according to claim 1, wherein an interior surface of the tubular body bore has a plurality of inwardly extending projections thereon, the inwardly extending projections being adapted to engage a lead body of an implantable surgical device.

4. The suture sleeve according to claim 1, wherein the tubular body is formed from reinforced silicon.

5. The suture sleeve according to claim 1, further comprising:
an inflatable balloon within the closed annular space.

6. A suture sleeve comprising:
a base;
a tubular body attached to the base; and
an inflatable balloon attached to the base and the tubular body, the balloon circumferentially extending around a portion of the tubular body.

7. The suture sleeve according to claim 6, wherein an undersurface of the base is substantially flat.

8. The suture sleeve according to claim 6, wherein the base and the tubular body form a unitary structure.

9. The suture sleeve according to claim 6, wherein the suture sleeve has a smooth exterior surface.

10. The suture sleeve according to claim 6, wherein the base has a plurality of attachment members therein.

11. The suture sleeve according to claim 10, wherein the attachment members are wings extending outward from the tubular body.

12. The suture sleeve according to claim 11, wherein each wing has at least one attachment aperture therein.

13. The suture sleeve according to claim 12, wherein each attachment aperture has a raised ring extending at least partially therearound.

14. The suture sleeve according to claim 6, wherein the balloon has an inflation port therein.

15. The suture sleeve according to claim 14, wherein the inflation port is self-sealing.

16. The suture sleeve according to claim 14, wherein the inflation port extends outwardly from the balloon.

17. The suture sleeve according to claim 6, wherein an interior surface of the tubular body has a plurality of inwardly extending projections thereon, the inwardly extending projections being adapted to engage a lead body of an implantable surgical device.

18. The suture sleeve according to claim 17, wherein the plurality of inwardly extending projections comprise pairs of opposed arcuate ridges, each arcuate ridge circumferentially extending less than 180°, a pair of arcuate ridges being longitudinally spaced from an adjacent pair of arcuate ridges.

19. The suture sleeve according to claim 6, wherein the suture sleeve is formed from reinforced silicon.

20. The suture sleeve according to claim 19, wherein the reinforced silicon is reinforced with nylon mesh.

21. A suture sleeve comprising:
a tubular body having a bore extending therethrough and an internal cavity circumferentially extending partially around the bore; and
an inflatable balloon within the tubular body internal cavity.

22. The suture sleeve according to claim 21, wherein the suture sleeve has a smooth exterior surface.

23. The suture sleeve according to claim 21, wherein the bore has a plurality of inwardly extending ridges thereon.

24. The suture sleeve according to claim 21, wherein the tubular body is formed from reinforced silicon.

25. The suture sleeve according to claim 21, wherein the balloon has an inflation port, the inflation port extending through an external surface of the tubular body.

26. The suture sleeve according to claim 25, wherein the inflation port is self-sealing.

27. The suture sleeve according to claim 21, wherein the bore is radially flexible.

28. The suture sleeve according to claim 21, wherein the balloon is filled with a saline solution.

29. The suture sleeve according to claim 21, wherein the tubular body includes a plurality of attachment members adapted to be secured to adjacent tissue.

30. The suture sleeve according to claim 21, wherein the suture sleeve is formed from reinforced silicon.

31. A suture sleeve comprising:
a reinforced silicon tubular body having: a flexible bore extending therethrough; an internal cavity circumferentially extending partially around the bore; and a plurality of attachment wings extending outward from the tubular body, the bore having a plurality of inwardly extending ridges thereon, the plurality of inwardly extending ridges comprising pairs of opposed arcuate ridges, each arcuate ridge circumferentially extending less than 180°, a pair of arcuate ridges being longitudinally spaced from an adjacent pair of arcuate ridges, a central portion of an undersurface of the tubular body being substantially flat; and
an inflatable balloon within the tubular body internal cavity, the balloon having a self-sealing inflation port, the inflation port extending through an external surface of the tubular body.

32. The suture sleeve according to claim 31, wherein each attachment wing has at least one attachment aperture therein.

33. The suture sleeve according to claim 32, wherein each attachment aperture has a raised ring extending at least partially therearound.

34. In combination:
a suture sleeve having a flexible bore extending longitudinally therethrough and an expandable closed annular space circumferentially extending at least partially around the bore;
a syringe; and
a predetermined amount of saline solution for filling the suture sleeve inflatable chamber.

35. The combination according to claim 34, further comprising:
a surgical stapler with a plurality of staples adapted to secure the suture sleeve to adjacent tissue.

36. The combination according to claim 35, wherein each staple has a central portion with a leg extending downward from each end of the central portion, the central portion and adjacent portions of each leg being coated with silicon.

37. The combination according to claim 34, wherein the syringe comprises: a fluid tube; a moveable plunger positioned within a first end of the fluid tube; a needle extending from a second end of the fluid tube; and a protective cone extending from the second end of the fluid tube, the protective cone extending about the needle.

38. The combination according to claim 37, wherein the fluid tube has a plurality of finger engaging projections thereon and the moveable plunger has a thumb ring on an end thereof.

39. The combination according to claim 34, wherein the predetermined amount of saline solution is within the syringe.

40. The combination according to claim 34, further comprising:
a pacer system including a pacer lead, the pacer lead extending through the suture sleeve bore.

41. The combination according to claim 34, wherein the suture sleeve has an inflatable balloon within the closed annular space.

42. A surgical staple comprising:
a central portion;
a leg extending downward from each end of the central portion, the central portion and the legs being monolithic; and
a silicon coating enclosing the central portion and adjacent portions of each leg.
